(19) United States
(12) Patent Application Publication
(10) Pub. No.: US 2024/0358250 A1
(43) Pub. Date: Oct. 31, 2024

(54) LIGHT GUIDE PIPE FOR UNIFORM ENDOSCOPIC ILLUMINATION

(71) Applicant: Covidien LP, Mansfield, MA (US)

(72) Inventors: Juergen Van Vlem, Tsim Sha Tsui (HK); Yik Loong Tham, Sha Tin (HK); Soren Aasmul, Holte (DK)

(73) Assignee: Covidien LP, Mansfield, MA (US)

(21) Appl. No.: 18/623,424

(22) Filed: Apr. 1, 2024

Related U.S. Application Data
(60) Provisional application No. 63/498,667, filed on Apr. 27, 2023.

(51) Int. Cl. A61B 1/267; A61B 1/04; A61B 1/06

(52) U.S. Cl. CPC A61B 1/267; A61B 1/04; A61B 1/0676

(57) ABSTRACT

The technology relates to video laryngoscopes with improved illumination. An example laryngoscope includes a display; a handle; an arm extending distally from the handle; a camera positioned at a distal end of the arm; a light source positioned at the distal end of the arm; and a unibody plug positioned at the distal end of the arm, the unibody plug. The unibody plug includes a light guide formed of a solid transparent material having an entry surface at a proximal end of the light guide and an exit surface at a distal end of the light guide, wherein the entry surface is adjacent the light source; and a frame that defines an air gap around at least 70% of a length of the light guide.

### CROSS-REFERENCE TO RELATED APPLICATIONS

This application claims the benefit of U.S. Provisional Application No. 63/498,667 filed Apr. 27, 2023, titled “Light Guide Pipe for Uniform Endoscopic Illumination,” which is incorporated herein by reference in its entirety.

### BACKGROUND

Video laryngoscopes are commonly used to perform intubations on patients who require breathing assistance. During an intubation, the video laryngoscope (VL) may be used to manipulate the anatomy of the larynx and associated structures of a patient's airway, in order to obtain a view sufficient for insertion of a breathing tube (e.g., an endotracheal tube) into the trachea. A VL includes a light source for illuminating the airway and a camera for collecting airway images, which are viewed on a display associated with the VL. The acquired image may facilitate insertion of the breathing tube.

It is with respect to this general technical environment that aspects of the present technology disclosed herein have been contemplated. Furthermore, although a general environment is discussed, it should be understood that the examples described herein should not be limited to the general environment identified herein.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter. Additional aspects, features, and/or advantages of examples will be set forth in part in the description which follows and, in part, will be apparent from the description, or may be learned by practice of the disclosure.

In an aspect, the technology relates to a video laryngoscope that includes a display; a handle; an arm extending distally from the handle; a camera positioned at a distal end of the arm; a light source positioned at the distal end of the arm; and a unibody plug positioned at the distal end of the arm, the unibody plug. The unibody plug includes a light guide formed of a solid transparent material having an entry surface at a proximal end of the light guide and an exit surface at a distal end of the light guide, wherein the entry surface is adjacent the light source; and a frame that defines an air gap around at least 70% of a length of the light guide.

In an example, the frame further defines a cavity for receiving the camera. In another example, the frame and the light guide are formed from the same transparent material. In still another example, a surface area of the entry surface is less than a surface area of the exit surface. In yet another example, a center of the exit surface is positioned anteriorly from a center of the entry surface. In still yet another example, at least one of the entry surface or the exit surface is textured to cause diffusion of light from the light source. In a further example, at least one of the entry surface or the exit surface is formed as a concave or convex lens.

In another aspect, the technology is directed to a unibody plug for guiding light emitting from a video laryngoscope. The unibody plug includes a light guide, formed of a solid transparent material, having an entry surface at a proximal end of the light guide and an exit surface at a distal end of the light guide, wherein the entry surface is adjacent a light source of the video laryngoscope; and a frame, formed of the solid transparent material, that defines an air gap around at least 70% of a length of the light guide.

In an example, the air gap is around at least 90% of the length of the light guide. In another example, the frame further defines a cavity for receiving a camera of the video laryngoscope. In yet another example, a center of the exit surface is positioned anteriorly from a center of the entry surface. In still another example, a surface area of the entry surface is substantially the same as a surface area of the exit surface. In a further example, a width and height of the entry surface is substantially the same as a width and height of the exit surface. In still yet another example, the air gap is filled with a material that has a refractive index different than the transparent material of the light guide.

In another aspect, the technology is directed to a video laryngoscope including a display; a handle; an arm extending distally from the handle; a camera positioned at a distal end of the arm; a light source positioned at the distal end of the arm; and a unibody plug positioned at the distal end of the arm, The unibody plug includes a light guide formed of a solid transparent material, the light guide including an entry surface at a proximal end of the light guide, the entry surface having a first center and a first surface area; an exit surface at the distal end of the light guide, the exit surface having: a second center positioned anteriorly from the first center of the entry surface; and a second surface area that is substantially the same as the first surface area of the entry surface. The unibody plug further includes a frame formed of the solid transparent material. The frame defines an air gap around at least 90% of a length of the light guide; and a cavity that receives the camera.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIGS. 1A-1B depicts views of an example VL that includes a light source and camera.

FIG. 2 depicts an enhanced view of the arm and blade of an example VL near the camera and light source.

FIG. 3 is an example ray diagram depicting concepts of refraction and total internal reflection.

FIGS. 4A-4B depicts views of the distal portion of an example arm.

FIG. 5 depicts an enhanced cross-sectional view of an example light source and light guide.

FIGS. 6A-6G depict example light guide variations.

FIGS. 7A-7D depict further example light guide variations.

### DETAILED DESCRIPTION

Patients who require breathing assistance may be connected to a mechanical ventilator via breathing tube (e.g., an endotracheal tube). In a medical procedure referred to as an intubation, a clinician inserts a breathing tube into the mouth of the patient, past the larynx, and into the trachea. The breathing tube may then be connected to a ventilator or other device for supplying breathing gases to the patient. A laryngoscope may be used during intubation to help the clinician manipulate portions of the patient's anatomy, such as the tongue and epiglottis, and obtain a view of the larynx sufficient for inserting the breathing tube into the trachea. To further help visualize the larynx, some laryngoscopes may be configured with a video camera. A laryngoscope that includes a video camera may be referred to as a video laryngoscope (VL).

The VL further includes a light source that illuminates a portion of the airway. In some VLs, the camera and light source are located together at the end of an extension or arm of the VL, which is navigated into the airway so that the larynx is within the field of view (FOV) of the camera. The light source may be coupled to a light guide that directs light emitted from the light source onto the FOV. The light guide may also be referred to as a light pipe because the role of the light guide is to connect (“pipe”) light from the light source to the airway. A typical light guide may include a transparent plastic tube surrounded by an opaque plastic housing. As light is directed through the light guide, the light interacts with the plastic housing, which may result in loss at the boundary between the light guide and the housing and may reduce the intensity of illumination.

Further, the light may be transmitted from the light guide into the airway in an uncontrolled manner. In one example, a portion of the light may reflect off surfaces of the VL blade and onto portions of the patient's anatomy, resulting in portions of the airway image being over-illuminated (e.g., bright spots or glare). In addition, a portion of the light transmitted from the light guide may be directed out of the FOV of the camera or may be reflected out of the FOV of the camera (such as by reflecting off surfaces of the blade and out of the FOV). Light directed away from the FOV may leave portions of the airway image under-illuminated. For instance, one or more edges of the captured image may appear dimmer than others and in general the lighting may be non-uniform.

The technology described herein relates to the design of a light guide in the form of a light pipe that improves uniformity of illumination of the airway image. Rather than a plastic housing, the light guide described herein is substantially surrounded by air, (or other material with a lower index of refraction than the lightguide) which promotes total internal reflection within the light guide. This approach helps reduce losses at the air/light guide boundaries. The surfaces that form the sides of the light guide may be curved or shaped to further promote total internal reflection, to efficiently direct light from the light source to the airway, and to reduce scattered light that may impact image quality. The surface where the light enters and exits the light guide may also be modified to improve illumination. For example, the exit surface of the light guide (where the light is transmitted into the airway) may be designed to focus light away from the blade and onto the FOV, which may both reduce bright spots and glare, and may reduce under-illumination of portions of the airway image. In some examples, the material of the light guide may be modified in localized regions, such as by texturing the material, in order to diffuse the light and provide a more even illumination of the airway image. The configurations of the light guides discussed herein may improve the uniformity and intensity of the illumination of the field of view of the camera. The light guide may also be fabricated from a single material, in a single-step process, such as by a molding process. Thus, the design of the light guide may also simplify the manufacturing process. Additional details are now provided by way of discussion of the accompanying drawings.

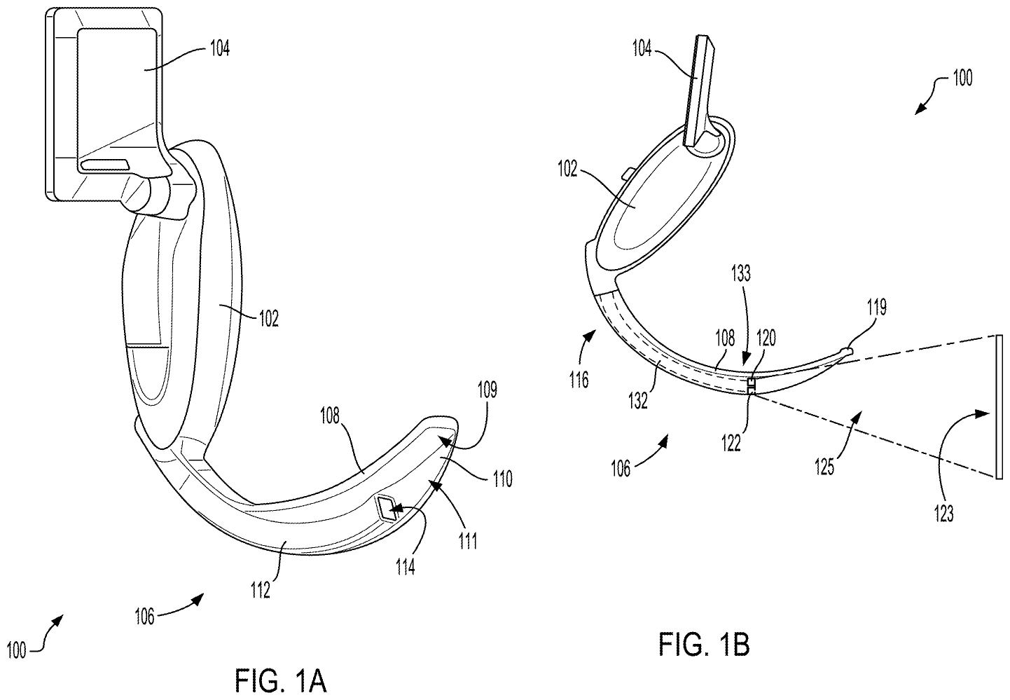

FIGS. 1A and 1B depict views of an example video laryngoscope (VL) 100 that includes a light source 120 and camera 122 for illuminating and imaging the airway, respectively. The example VL 100 includes a handle 102, a display 104, and blade 106 that may be removably attached to a hollow extension or arm 132 that extends distally from the handle 102. The light source 120 and camera 122 are housed within the arm 132 and may be affixed within the distal portion 133 of the arm 132. The arm 132 may be permanently affixed to the handle 102, and in some examples may be referred to as a “camera stick.” The handle 102 may include a power source (e.g., battery), processor, memory, and/or other electrical elements needed to operate the light source 120 and receive image data from the camera 122.

Image data from the camera 122, such as an airway image 123, may be displayed on the display 104. The display 104 may be any of a variety of display technologies, such as LCD, LED, OLED, or other display technology. In examples, the display 104 may be a touch-sensitive display (e.g., a capacitive touch-sensitive display) that allows user input to be received through the display 104. For example, the light source 120 and camera 122 may be controlled and/or configured via touch input received from the user through the display 104. In other examples, the light source 120 and camera 122 may be controlled and/or configured by other user input methods, such as by buttons, switches, or other input elements provided by the example VL 100.

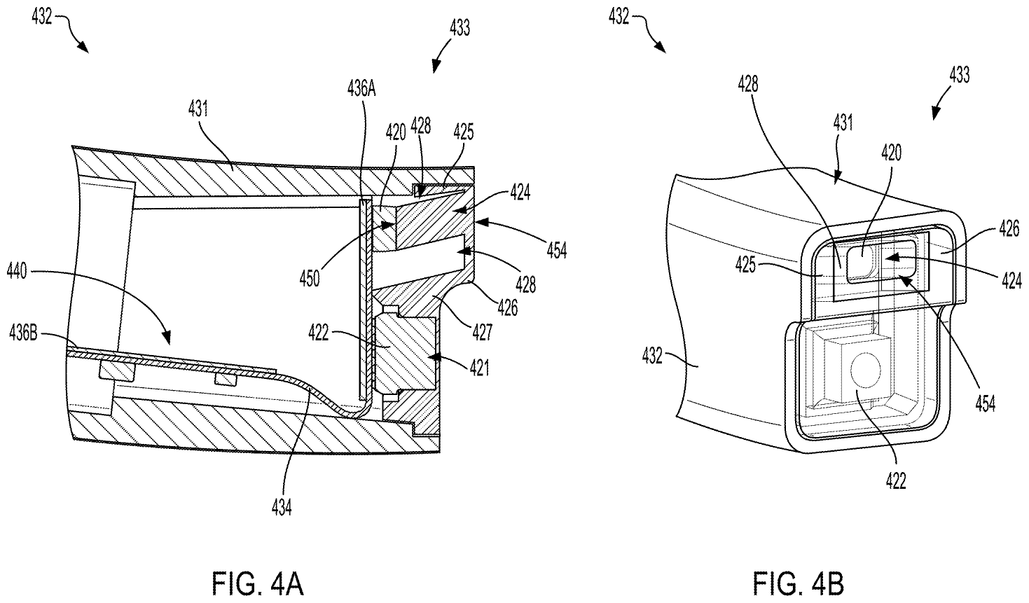

The light source 120 and camera 122 are connected to electrical elements in the handle 102 and/or display 104 by electrical conductors that are routed through the arm 132. The electrical conductors may include one or more wires, flexible printed circuits (FPCs), and/or other types of electrical conductors. An example where the light source 120 and camera 122 are mounted to an FPC is depicted in FIG. 4A. In examples, the light source 120 may be an LED or other type of light source suitable for being positioned within the distal portion 133 of the arm 132. The camera 122 may be a type of digital camera or sensor capable of taking video and/or still images of the airway and suitable for being positioned within the distal portion 133 of the arm 132.

The blade may also include a window or lens 114, through which the light source 120 illuminates the airway and the camera 122 may acquire the airway image 123. The curvature of the arm 132 and blade 106, along with the positioning of the window or lens 114, are designed to orient the field of view (FOV) 125 of the camera 122 towards the distal tip 119 of the blade 106, in order for the camera 122 to acquire the airway image 123.

The FOV 125 may also intersect portions of the blade 106, which are captured as part of the airway image 123 and displayed. For example, the blade 106 may include a flange 108 used to manipulate the patient's anatomy. The FOV 125 may include a portion of the superior or camera-facing surface 109 of the flange 108. In addition, the blade 106 also includes a wall 110 that serves as a structural element of the blade 106. The FOV 125 may encompass a lateral surface 111 of the wall 110, which is also captured as part of the airway image 123.

In some examples, the blade 106 may be detachable from the handle 102. For example, the proximal end 116 of the blade 106 may detach from the handle 102 and slide away from, and off of, the arm 132. The handle 102 and display 104 are cleaned between uses, and the blade 106 may be detached and discarded or cleaned between uses. The handle 102 and arm 132 may be designed to accept a variety of types of blades 106, such as blades 106 with different curvatures. For instance, a clinician may select a blade 106 that is substantially flat or flat, substantially curved (such as the depictions in FIGS. 1A-B), or that includes a combination of one or more curvatures along the length of the blade 106. In other examples, the example VL 100 may include a blade 106 with a defined curvature that is permanently affixed to the handle 102.

In addition, the blade 106 may be fabricated from metal, plastic, or a composite material suitable for forming a blade 106. For example, the blade 106 may be made of a transparent or translucent plastic. The surfaces of the blade 106 (such as inferior surface 109 and lateral surface 111) may be substantially smooth, which may facilitate insertion of the blade 106 into the airway and cleaning of the blade 106. The material from which the blade 106 is fabricated, the smoothness of the blade 106, and/or other factors associated with the blade 106 affect the specularity of the blade surfaces. For example, a blade 106 with a smooth or polished surface may reflect light generated by the light source 120 onto the airway image 123. Light reflected from one or more surfaces of the blade 106 may result in portions of the airway image 123 being over-illuminated, such as by the occurrence of bright spots.

In examples, the camera 122 is positioned farther from the flange 108 than the light source 120 to reduce the portion of the flange superior surface 109 that occupies the FOV and airway image 123. With the light source 120 closer to the superior surface 109, reflected light, if left uncontrolled, may cause further over-illumination of the airway image 123. A position closer to the flange 108 when in use may be considered to be a more anterior or inferior position, and a position further from the flange 108 may be considered a more posterior or superior position. Accordingly, in the example depicted, the camera 122 is positioned posteriorly from the light source 120.

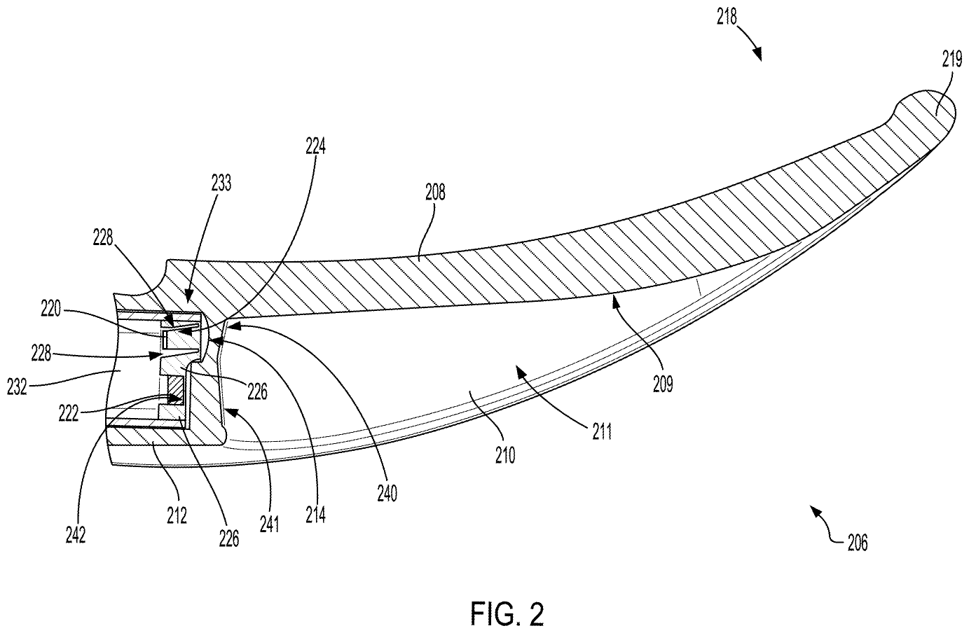

FIG. 2 depicts a cross-sectional view of a distal end of an arm 232 with an example blade 206. The example blade 206 includes a flange 208 with a superior surface 209, and a wall 210 with a lateral surface 211. The blade 106 further includes a channel 212, which houses the arm 232. The distal end of the arm 232 is depicted in FIG. 2 and includes a light source 220 and camera 222, which are directed towards the distal tip 219 through a lens 214 of the channel 212.

The arm 232 includes a transparent unibody structure in the form of a unibody plug 226 that plugs the distal portion 233 of the arm 232. The unibody plug 226 may contact the walls of the arm 232 and seals internal portions of the arm 232. For example, the unibody plug 226 may be sealed against the arm 232 by adhesive or by another material suitable for forming a seal. The seal between the unibody plug 226 and arm 232 protects the light source 220, camera 222, and other elements from exposure to environmental hazards, such as the ingress of saliva from the patient's airway, cleaning fluids, and other fluids or hazards that may contact the unibody plug 226 and arm 232.

In examples, the unibody plug 226 is a single, continuous element, that may be formed in a single-step molding process from a single material. For instance, the unibody plug 226 may be made of a transparent plastic, glass, composite, or other type of transparent material. The camera 222 images the airway through the unibody plug 226, and the light source 220 illuminates the FOV through the unibody plug 226.

The unibody plug 226 includes a light guide 224 that channels the light generated by the light source 220 through the unibody plug 226 and lens 214 and onto the FOV. The lens 214 may be shaped or otherwise configured to modify the light projected onto the FOV. One or more surface of the lens 214 may include an anti-reflective coating. The surfaces 240, 241, 242 that form optical interfaces (e.g., plastic/air interfaces) may also include an anti-reflective coating. The anti-reflective coating may reduce reflections in the boundary of about 4% per surface in some examples. These anti-reflective coatings may improve light output by up to about at least 8% and further increase light received by the camera by up to about at least 8%. A reduction in light reflected at the boundaries also results in a reduction in glare.

The light guide 224 is oriented in a substantially proximal-to-distal direction. As described below, the light guide 224 includes features that may improve the quality of illumination of the FOV. For example, the unibody plug 226 includes an air gap 228 surrounding the light guide 224. The air gap 228 is described in greater detail below, with respect to FIG. 4. The ratio of the index of refraction of air in the air gap 228 to the index of refraction of the unibody plug 226 establishes boundary conditions that promote total internal reflection within the light guide 224. In addition, the light guide 224 may be shaped such that light exiting the light guide 224 is directed substantially away from the flange superior surface 209 and towards the FOV, thereby reducing glare and/or over-illumination of portions of the airway image.

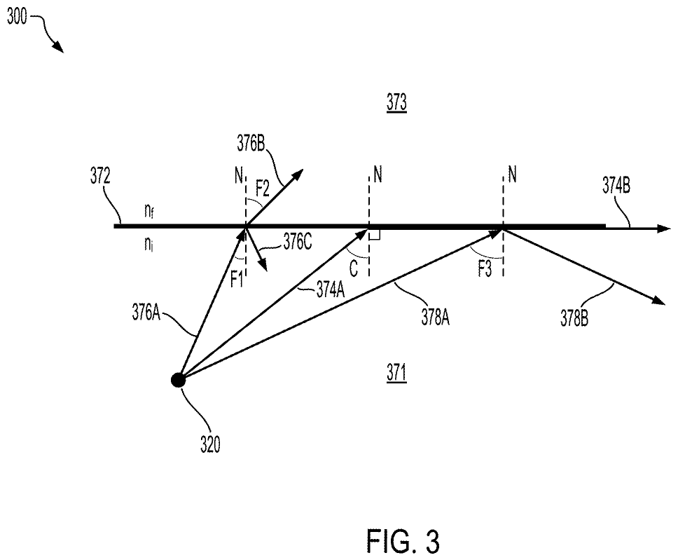

FIG. 3 is an example ray diagram 300 depicting a set of light rays 374A, 376A, and 378A incident upon a boundary 372 between a first medium 371 and an air medium 373. The example ray diagram 300 depicts concepts associated with the optical phenomenon of total internal reflection. The first medium 371 includes a light source 320 from which the rays 374A, 376A, and 378A are emitted. Although the light source 320 is depicted as a point source, the light source 320 may be representative of a larger, distributed light source (such as light source 220) that emits light rays that are incident upon a boundary from a multitude of incident angles.

The material of the first medium 371 may be similar to, or the same as, the material of the unibody plug 226. For example, the first medium 371 may be a type of plastic, glass, composite, or another material that has a higher optical density than air and that is suitable for forming the unibody plug 226 and the light guide 224. The first medium 371 is transparent or substantially transparent at visible wavelengths and has an index of refraction, nᵢ. In examples where the first medium 371 is a type of plastic or glass, the index of refraction may be approximately 1.5. The air medium 373 has an index of refraction, nᵣ, typically approximated as 1.0.

A first ray 374A is incident upon the boundary 372 at an angle C. The boundary 372 represents the interface between the first medium 371 and the air medium 373. The angle C corresponds to the critical angle, which is the incident angle at which light is refracted along the boundary 372 at a 90° angle from a line N normal (perpendicular) to the boundary 372. The first ray 374A is refracted as ray 374B. The critical angle C may be calculated according to Eq. (1) using the indices of refraction defined above for the first medium 371 and the air medium 373.

$$C = {sin}^{- 1} ( \frac{{n}_{f}}{{n}_{i}} ) \qquad Eq . ( 1 )$$

In examples where the values of the indices of refraction are approximately as provided above, the critical angle for light incident upon the boundary 372 from within the first medium 371 is approximately 42°. In other examples, the index of refraction of the first medium 371 may be greater or less than 1.5 (e.g., depending on the material of the unibody plug 226), resulting in a critical angle C that is greater or less than approximately 42°.

A second ray 376A may be incident upon the boundary 372 at an angle F1 that is less than the critical angle C (relative to a normal line N). Upon impinging the boundary 372, a portion of the light from the second ray 376A reflects back into the first medium 371 as ray 376C. A portion of the light from the second ray 376A is refracted at the boundary 372 as ray 376B. The angle F2 at which the ray 376B departs from the first medium 37 is larger than the incident angle F1.

A third ray 378A may be incident upon the boundary 372 at an angle F3 that is greater than the critical angle C (relative to a normal line N). At such an angle, the third ray 378A is totally reflected back into the first medium 371 as ray 378B, and no portion of the third ray 378A is refracted into the air medium 373. Light rays approaching the boundary 372 at angles greater than the critical angle C are similarly reflected back into the first medium 371. Rays reflected back into the first medium 371 may be subsequently reflected off of other interfaces between medium 371 and air medium 373. This principle may be applied to light generated by a light source (such as light sources 220) traveling within the light guide(s) described herein.

Returning now to the light source and light guide associated with a VL system, FIG. 4A depicts an enhanced cross-sectional view of an example arm 432, and FIG. 4B depicts a perspective view of the distal end of the example arm 432. The example arm 432 and associated elements may be similar to, or the same as, arm 232 depicted in FIG. 2. For instance, the example arm 432 includes a light source 420, camera 422, and unibody plug 426, which may be similar to, or the same as, corresponding elements described with respect to FIG. 2.

As depicted in FIG. 4A, the light source 420 and camera 422 are mounted to a circuit board, such as FPC 434, that electrically connects the light source 420 and camera 422 to electrical elements associated with a VL (e.g., example VL 100), such as in a handle (e.g., handle 102) and/or display (e.g., display 104) of the VL. The FPC 434 may be further connected to a first substrate 436A that may provide mechanical reinforcement to the FPC 434 in the area of the light source 420 and camera 422. The FPC 434 may also include a component region 440 that may include active or passive electrical components that support the operation of the light source 420 and camera 422, or that may be associated with another function or feature of the VL. The component area 440 may also be supported by a second substrate 436B.

The distal portion 433 of the example arm 432 includes the unibody plug 426, which caps the distal portion 433 of the example arm 432 and may be sealed against the walls of the arm 432, as described above. The camera 422 captures images of the airway through a portion of the unibody plug 426, which is transparent or substantially transparent. A portion of the unibody plug 426 forms a light guide 424 that directs light from the light source 420 to an exit surface 454, where the light is transmitted from the unibody plug 426 to the patient's airway to illuminate the FOV. The light guide 424 may be made as a solid piece of the transparent material of the unibody plug 426. Light is provided into the light guide 424 from the light source 420 at entry surface 450, which may be in contact with or adjacent to the light source 420. The entry surface 450 and of the exit surface 454 of the light guide 424 may be substantially rectangular shaped with rounded corners (such as depicted in FIG. 4B), while in other examples the light guide 424 may have a different shape, such as a substantially circular shape, oval shape, or other shape.

An air gap 428 surrounds a portion of the exterior of the material of the unibody plug 426 forming the light guide 424. The unibody plug 426 includes additional structures or protrusions that define the air gap 428 around the majority of length of the light guide 424. For instance, the air gap 428 may surround more than 60%, 70%, or 80% of the length of the light guide 424 between the light source 420 and exit surface 454. In other examples, the air gap 428 may surround more than 90% or 95% of the length of the light guide 424. The more of the light guide 424 that is surrounded by the air gap 428, the more efficient the internal reflection conditions may be, which results in brighter overall illumination. As depicted in FIG. 4B, the air gap 428 is circumferentially continuous around the exterior of portions of the light guide 424 and may be defined by surfaces of the unibody plug 426 and/or portions of the walls of the example arm 432. For instance, the air gap 428 may be substantially annular or ring shaped around the light guide 424. The air gap 428 may by partially conical in some examples as well. The air gap 428 may progressively narrow in the distal direction on the top side, whereas the air gap 428 may expand on the bottom side. The air gap 428 may have a thickness or depth on the sides that is substantially consistent. With different configurations of the light guide 424, the shape of the air gap 428 may change as well.

For example, the unibody plug 426 includes the light guide 424 and additional portions that support the light guide 424, and in some examples, the camera 422 as well. In the example depicted, the unibody plug 426 includes a frame 425 that forms the outer or exterior structure of unibody plug 426. The frame 425 is a portion of the unibody plug 426 and may be formed of the same material as the light guide 424. For instance, the exterior surfaces of the frame 425 may contact the interior surfaces of the arm 432 of the VL when the unibody plug 426 is installed in the arm 432. The material of the anterior/inferior portion of the frame 425 surrounds the light guide 424 offset by the air gap 428. Accordingly, the air gap 428 may be defined by the space between an interior surface of the frame structure 425 and the exterior surface of the light guide 424. Because the unibody plug 426 may be formed of a single material, the frame 425 is formed of the same material as the light guide 424.

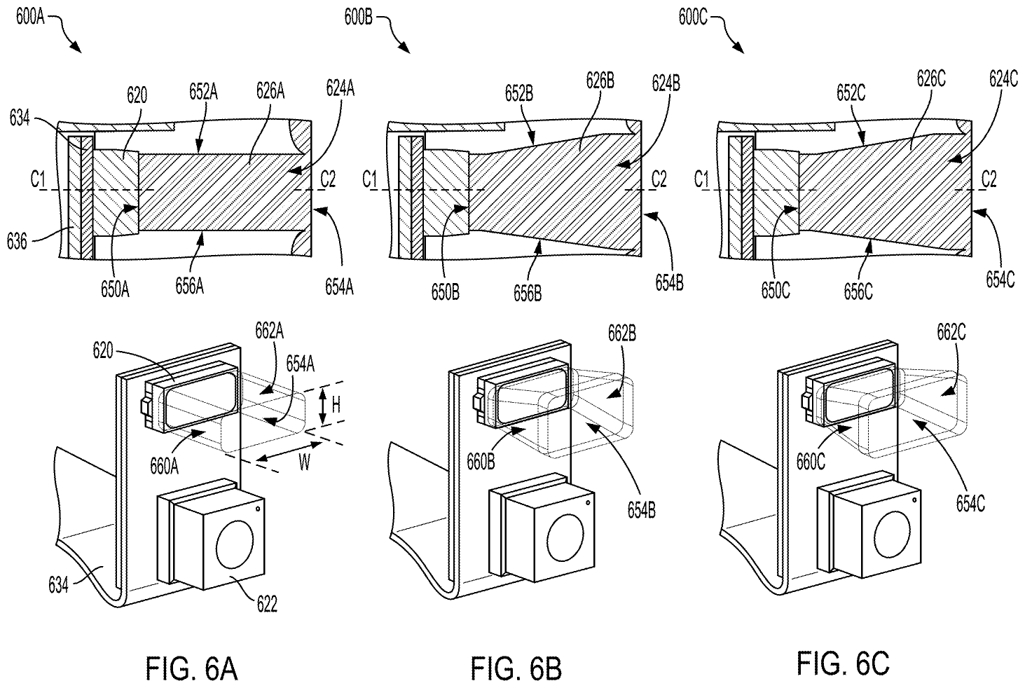

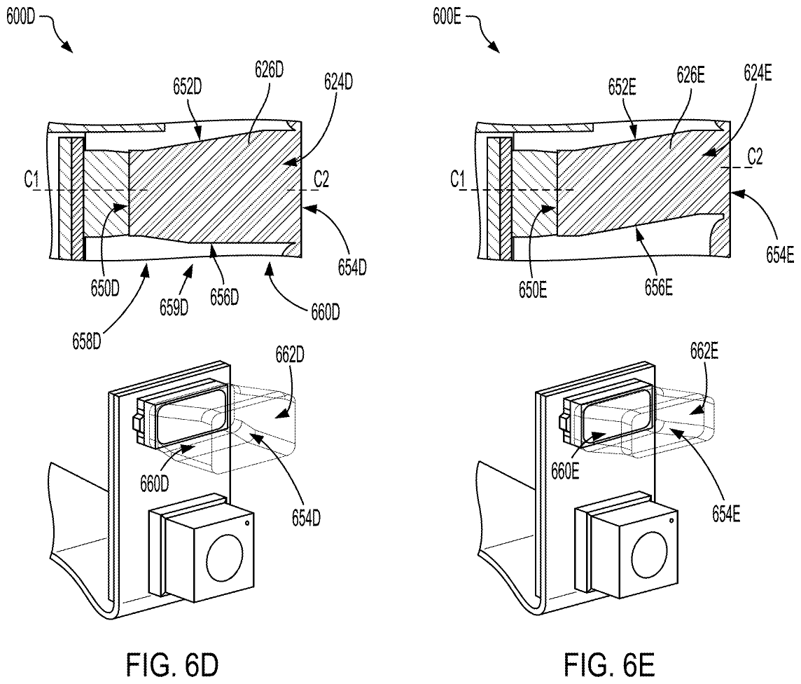

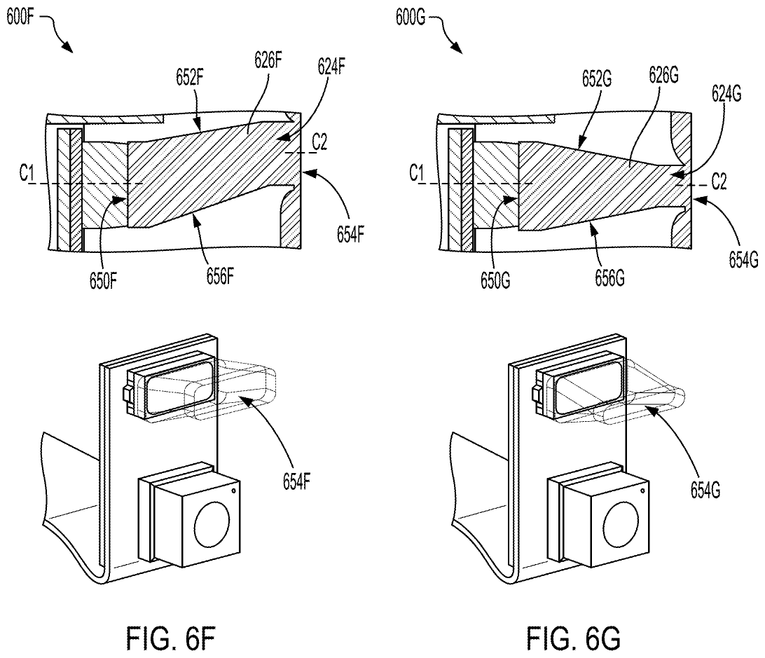

Depending on the shape and orientation of the light guide 424, the air gap 428 may be thinner, smaller, or may occupy less volume near some portions of the light guide 424 than near other portions of the light guide 424. For example, the air gap 428 may be thinner/smaller or may taper or narrow near portions of the light guide 424 that approach an inferior or anterior wall 431 and/or the frame 425. The air gap 428 may be larger near other portions of the light guide 424, such as between the light guide 424 and camera 422. In other examples, the air gap 428 may be partially or substantially symmetric around the exterior of the light guide 424, such as where the surfaces of the light guide 424 are symmetric, and where the frame 425 and other surfaces that define the air gap 428 are configured to provide symmetry. Additional cross-sectional views of the light guide 424 and portions of the air gap 428 are provided in FIGS. 6A-G, in which a variety of example light guide and air gap configurations are depicted.

Further, the air gap 428 introduces boundary conditions along the length of the light guide 424 suitable for promoting total internal reflection of light emitted from the light source 420 and improving illumination of the airway. For example, the presence of the air gap 428 increases the amount of light retained in the light guide 424 due to the effects of total internal reflection and reduces the amount of light refracted out of the light guide 424. Light retained in the light guide 424 may then be directed to the FOV of the camera 422, which may improve the intensity of illumination of the airway image may improve the efficiency of the light guide 424. In some examples, the light guide 424 may be referred to as a light pipe, due to the ability of the light guide 424 to effectively retain light received at the entry surface 450 and direct it to the exit surface 454 (as a result of total internal reflection, among other things). While described herein as an air gap 428, in some examples, the air gap 428 may be filled with another material that has a substantially different index of refraction as the material of the light guide 424 such that the internal reflection properties discussed herein are retained.

In addition, light that escapes from the light guide 424 prior to exiting through the exit surface 454 may be reflected and/or scattered by internal structures and surfaces of the unibody plug 426 and other elements of the example arm 432. In some scenarios, this escaped light may enter the lens structure of the camera 422, resulting in lens flare or other unwanted optical effects. By improving total internal reflection along the length of the light guide 424 and helping retain more light within the light guide 424, the air gap 428 may reduce the light that escapes from the light guide 424 (increases retained light within the light guide 424), thereby reducing the effects of internally scattered light.

To avoid escaped light from entering the camera 422 the frame 425 can be masked by means of etching, texturizing, painting, coating, and/or making other modifications to any of the surfaces making up frame 425. Such modifications may absorb the escaped light or may direct/scatter/couple light out of the frame 425 to areas other than the camera lens.

To further reduce internally scattered light, the light guide 424 may be oriented to direct light from the light source 420 away from the camera 422. For instance, the light guide 424 may be oriented or tilted such that the exit surface 454 is positioned closer to the inferior or anterior wall 431 and farther from the camera 422. This arrangement may also reduce scattering by external elements, such as by saliva or extraneous portions of the patient's anatomy, that may be near the camera 422 but are not relevant to the airway image. These external elements may reflect light back towards the camera 422 and introduce unwanted optical effects. By positioning the exit surface 454 farther from the camera 422, reflections from external elements may be reduced.

In addition, one or more lenses (not depicted) may be included at the entry surface 450 and/or the exit surface 454. The lens(es) direct the light that propagates into the light guide 424 and/or out of the light guide 424. By directing the light from the light source 420 into the light guide 424 with a lens, the internal reflection properties of the light guide may be improved. By directing light from the light guide into the body by a lens at the exit surface 454, glare from the blade may be reduced and the light may be more precisely directed at the FOV of the camera 422. Alternatively to the inclusion of separate lenses, the entry surface 450 and/or the exit surface 454 may be shaped so as effectively form a lens. For instance, the entry surface 450 and/or the exit surface 454 may have a convex or concave curvature rather than being flat. The entry surface 450 and/or the exit surface 454 may also be etched, textured, painted, coated, or otherwise modified to diffuse the light propagating through the respective surface. The entry surface 450 and/or the exit surface 454 (or portions thereof) may also be provided with an anti-reflective coating to reduce unwanted reflection of light at these interfaces and to consequently enhance the effective transmission of light at these interfaces. In some examples, the anti-reflective coating may improve the efficiency of within the range of 4%-8% or greater.

The void between the front face of the light source 420 and the entry surface 450 may be filled with an index-matching substance such as optical adhesive, transparent UV curable adhesive, epoxy adhesive, an elastic gel or the like to reduce unwanted reflection of light at these interfaces and to consequently enhance the effective transmission of light at this interface. In examples, the index-matching substance has a refractive index that is an average (or an approximate average) between the refractive index of the light source 420 and the entry surface 450. In other examples, the index-matching substance has a refractive index that is between the refractive index of the light source 420 and the entry surface 450. The substance (e.g., adhesive) may be applied during assembly for subsequent curing.

In some examples, an elastic gel may be applied to either the entry surface 450 or front face of the light source 420. The elastic gel may be compressed by the assembly and fill the void for index matching. The gel may further have a slightly convex surface after being coated onto either the front surface of the light source 420 or the entry surface 450 to prevent air pockets being trapped between the gel and the opposite optical surface.

The frame 425 may also define cavity 421 for receiving the camera 422. For instance, the frame 425 may include a posterior portion or half that is positioned posteriorly from the light guide 424. The posterior portion of the frame 425 includes or defines a cavity that is shaped to receive the camera 422. A separation segment 427 of the frame 425 may be positioned between the air gap 428 and the cavity 421. Accordingly, the separation segment 427 may define a portion of both the air gap 428 and the cavity 421. By having the frame 425 define both the light guide 424 and the cavity 421 for the camera 422 within the same unibody plug 426, the single structure may be used to secure and protect both the light source 420 and the camera 422.

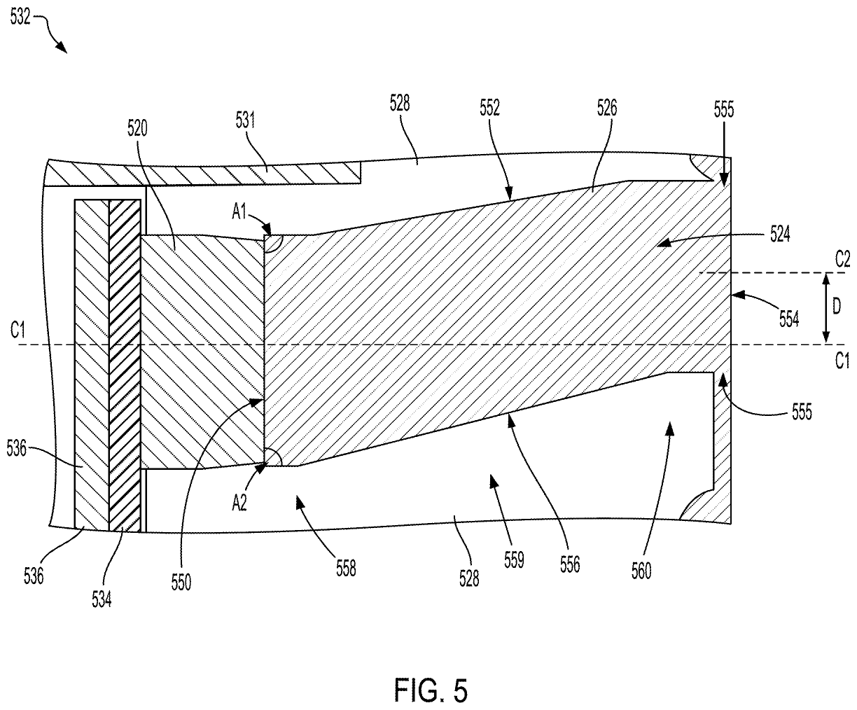

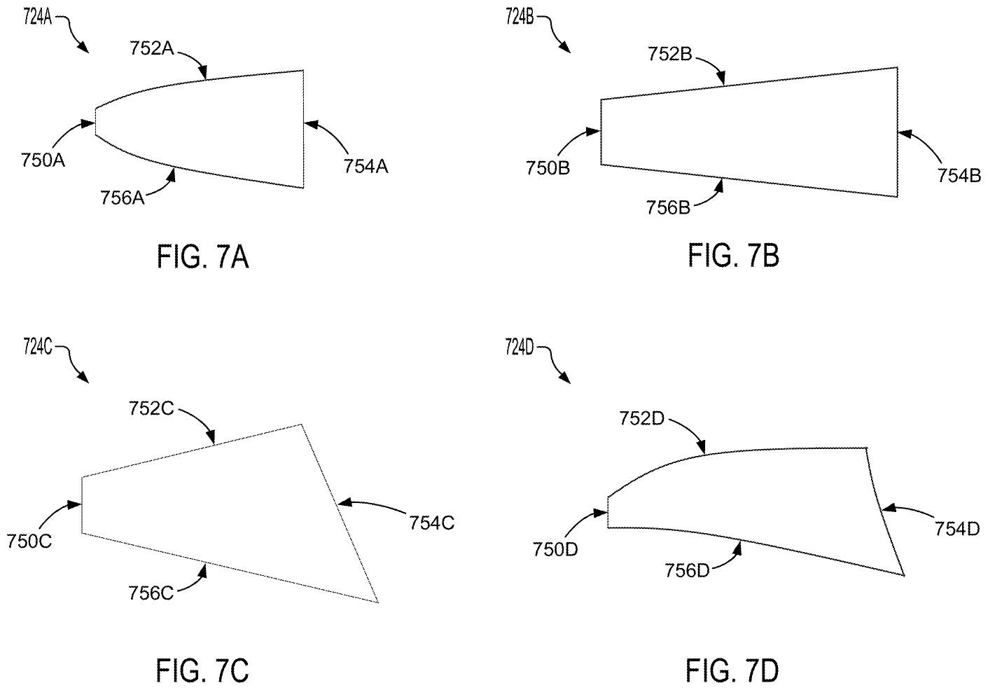

FIG. 5 depicts an enhanced cross-sectional view of a distal end of an example arm 532 in the region near a light source 520 and light guide 524 of the unibody plug 426. The example arm 532 and associated elements may be similar to, or the same as, example arm 432 and elements associated therewith. The light guide 524 depicted in FIG. 5 is one example of a number of possible design variations for implementing the light guide 524. Additional variations are depicted in FIGS. 6 and 7.

The light source 520 may be mounted to an FPC 534, which provides electrical power to the light source 520, such as electrical power provided by a power source in the VL. The FPC 534 may further be connected to a substrate 536 that may provide mechanical reinforcement to the FPC 534.

The entry surface 550 of the light guide 524 may be in contact with, or adjacent to, the light source 520. The entry surface 550 receives light emitted from the light source 520 into the light guide 524. In some examples, in addition to the air gap 528, air may be present between the entry surface 550 and light source 520, or the interface between the entry surface 550 and light source 520 may not be a sealed interface. In examples, the dimensions of the entry surface 550 may be substantially equal to the dimensions of the adjacent light emitting surface of the light source 520. For instance, the surface area of the entry surface 550 may be substantially equal to the surface area of the light emitting surface of the light source 520, which may promote light coupling from the light source 520 through the entry surface 550 and into the light guide 524. In some examples, the dimensions of the entry surface 550 may be larger or smaller than the light emitting surface of the light source 520.

As depicted in FIG. 5, the exit surface 554 may be offset vertically (e.g., in the inferior/anterior direction) from the entry surface 550, such that the exit surface 554 is positioned anteriorly farther from a camera of the example arm 532 (such as depicted in FIGS. 4A-B). For example, the center C2 of the exit surface 554 may be offset a distance D above the center C1 of entry surface 550. In other examples, the center C2 of the exit surface 554 may be offset below the center C1 of the entry surface 550 or may be substantially horizontally aligned with center C1.

The top (anterior/inferior) surface 552 and bottom (posterior/superior) surface 556 of the light guide 524 may be oriented and/or shaped to promote total internal reflection within the light guide 524 and to control how the light is directed from the light source 520 toward the exit surface 554. The top and bottom surfaces 552, 556 may be substantially parallel and straight over a substantial portion of the length of the light guide 524 between the entry surface 550 and exit surface 554. In a proximal region 558 the top surface 552 may form an angle A1 with the entry surface 550, and the bottom surface 556 may for an angle A2 with the entry surface 550. The angles A1, A2 may affect the amount of light coupled into, and retained within, the light guide 524, based on total internal reflection.

In some examples, in the proximal region 558, the top and bottom surfaces 552, 556 may be oriented toward the center C1 of the entry surface 550 such that both angles A1 and A2 are less than 90°. This orientation may reduce total internal reflection in the proximal region 558, since a larger portion of the light emitted from the light source 520 may be incident upon the top and bottom surfaces 552, 556 at angles less than the critical angle. Such an orientation may increase the light that escapes the light guide 524 in the proximal region 558 due to refraction at the top and bottom surfaces 552, 556. Conversely, in other examples, the top and bottom surfaces 552, 556 may diverge from one another (away from center C1) in the proximal region 558, such that angles A1 and A2 are both greater than 90°. This orientation may increase total internal reflection in the proximal region 558, since a greater portion of the light emitted from the light source 520 may be incident upon the top and bottom surfaces 552, 556 at angles greater than the critical angle. As depicted in FIG. 5, both angles A1 and A2 may be approximately 90°, which may improve total internal reflection (and reduce light leaked from the proximal region 558) compared to examples where angles A1 and A2 are less than 90°, but may provide a lesser degree of total internal reflection compared to examples where the angles A1 and A2 are greater than 90°.

Further, the top and bottom surfaces 552, 556 in the middle region 559 of the light guide 524 may also be oriented and shaped to promote total internal reflection and to steer light toward the exit surface 554. The top and bottom surfaces 552, 556 are depicted in FIG. 5 as being substantially straight and parallel in the middle region 559. As described above, the top and bottom surfaces 552, 556 are oriented to direct light away from the camera, which may reduce unwanted optical effects due to scattered light being transmitted into the lens structure of the camera. In other examples, the top and bottom surfaces 552, 556 may be arranged in a different orientation and/or with a different shape. In one example, the top and bottom surfaces 552, 556 may diverge from one another and away from center C1, such that the light guide 524 is substantially funnel shaped (e.g., FIG. 7B), with a larger exit surface 554 than depicted in FIG. 5. Such an arrangement may improve total internal reflection since a larger portion of light from the light source 520 may be incident upon the top and bottom surfaces 552, 556 at angles greater than the critical angle. In other examples, the top and bottom surfaces 552, 556 may converge near the exit surface 554, toward center C2 of the exit surface 554, such that the light guide 524 is substantially cone-shaped (e.g., FIG. 6G), with an exit surface 554 that is smaller than the entry surface 550. Such an arrangement may reduce total internal reflection since a larger portion of light may be incident upon the top and bottom surfaces 552, 556 at angles less than the critical angle. Several examples of different orientations of the top and bottom surfaces 552, 556 are provided in FIG. 6.

In still other examples, the top and bottom surfaces 552, 556 may be shaped to further affect total internal reflection and/or the direction of light propagating through the light guide 524. For instance, rather than being substantially straight in the middle region 559, the top and bottom surfaces 552, 556 may be curved and/or may include one or more corners at which the top and/or bottom surfaces 552, 556 change orientation. In one example, the top and bottom surfaces 552, 556 may follow a mathematically defined curve, such as a parabolic curve, exponential curve, or other type curve. In other examples, the top and bottom surfaces 552, 556 may be shaped differently from one another, such that the top surface 552 may have a particular shape or curve, and the bottom surface 556 may have a different shape or curve. Several examples of surface curvatures of the top and bottom surfaces 552, 556 are provided in FIG. 7, and are described in more detail below.

The entry surface 550 and exit surface 554 are depicted in FIG. 5 as substantially straight and parallel to one another. In examples, the entry surface 550 and/or exit surface 554 may also be shaped to further control light into and out of the light guide 524, respectively. For example, the entry surface 550 may be shaped as a convex lens that causes light rays entering the light guide 524 through the entry surface 550 to converge towards the center C1 of the entry surface 550. Alternatively, the entry surface 550 may be shaped as a concave lens that causes light rays entering the light guide 524 through the entry surface 550 to diverge away from the center C1 of the entry surface 550 and toward the top and bottom surfaces 552, 556. In other examples, the entry surface 550 may have another shape or may follow a defined curve to direct light into, and retain light within, the light guide 524. The shape of the entry surface 550 may be designed to complement the shape and/or or orientation of the top and bottom surfaces 552, 556, such as to promote total internal reflection within the light guide 524. As an example, the entry surface 550 may be shaped as a concave lens that causes light rays passing through the entry surface 550 to diverge toward the top and bottom surfaces 552, 556, and the top and bottom surfaces 552, 556 may also be designed to diverge from one another and from the center C1 between the entry and exit surfaces 550, 554, in order to promote total internal reflection.

Similarly, the exit surface 554 may be shaped to control the convergence/divergence of light leaving the light guide 524. As an example, an exit surface 554 that is substantially flat may allow light rays to diverge outside of the camera FOV, such as in examples where the top and bottom surfaces 552, 556 direct the light through the exit surface 554 in a divergent pattern. An exit surface 554 that is convex may, however, cause the light rays to converge substantially onto the FOV as the light rays pass through the exit surface 554. Thus, an exit surface 554 may be shaped to increase the amount of light illuminating the FOV and/or to direct a portion of the light away from reflective surfaces of the VL blade (such as blade 106). Further, the shape of the exit surface 554 may improve uniformity of illumination of the FOV. For instance, light that would otherwise be directed out of the FOV by a straight exit surface 554 may be directed onto the FOV by a curved exit surface 554, which may improve dark or under-illuminated regions near the edges of the FOV.

In addition, the entry surface 550 and/or exit surface 554 may be tilted relative to the middle region of the light guide 524. For example, the exit surface 554 may be oriented/tilted relative to the light guide 524 to cause incident light directed from the light guide 524 to be refracted away from the flange (such as flange 108) and toward the FOV of the camera. FIG. 7C, discussed further below, depicts a light guide where the exit surface is tilted at an oblique angle.

The light guide 524 may include additional features that improve the uniformity of illumination of the FOV. For instance, the material of the unibody plug 526 adjacent to the exit surface 554 (on the interior of the light guide 524) may be modified to cause diffusion of the light passing through the exit surface 554. In one example, texturing may be applied to the subsurface region 555 of the unibody plug 526, adjacent to, and along, the exit surface 554, to cause diffusion of the light. The process of texturing the material of the unibody plug 526 may include using a focused laser (or other method) to introduce defects to the material in the subsurface region 555. In other examples, the texturing may be provided by another layer or coating of material. The defects cause intentional scattering of the light, so that the light becomes more diffuse as it enters the airway. The degree of texturing may be controlled so as to control the level of light diffusion. For example, the density of defects or amount of texturing in the subsurface region 555 may be increased, or texturing may be introduced deeper into the unibody plug 526 so that the subsurface region 555 expands toward the entry surface 550.

One advantage of introducing texturing within the subsurface region 555 of the exit surface 554 is that the exit surface 554 itself, which is exposed to the airway, may remain smooth and easy to clean. Texturing applied directly to the exit surface 554 may trap dirt, pathogens, or other hazards that may be more difficult to remove via surface cleaning.

In other examples, an optical film may be applied to the exit surface 554 to cause diffusion of the light. The light may allow the exit surface 554 to be cleaned, while providing a level of diffusion. In still other examples, an optical coating may be applied to the exit surface 554 to provide a level of diffusion.

FIGS. 6A-6G depict views 600A-G of example light guides 624A-G defined by unibody plugs 626A-G, where the design of each light guide 624A-G has been varied to affect illumination of the FOV. Several of the elements depicted in FIG. 6A are substantially the same as corresponding elements depicted in the remaining FIGS. 6B-G. For example, the light source 620, camera 622, FPC 634, and substrate 636 depicted in FIG. 6A are substantially the same in the remaining FIGS. 6B-G. To aid visualization, these elements are not indicated in FIGS. 6B-G. With the exception of the example variations described below for the light guides 624A-G, elements depicted in FIGS. 6A-G may be similar to, or the same as, corresponding elements of FIGS. 4A-B and FIG. 5.

The example light guide 624A depicted in FIG. 6A includes a top surface 652A and bottom surface 656A that are substantially parallel to one another and straight. The entry surface 650A and exit surface 654A are also substantially parallel to one another and are substantially aligned horizontally, such that the center C1 of the entry surface 650A is substantially aligned horizontally with the center C2 of the exit surface 654A. Side surfaces 660A and 662A are also visible in the view 600A and are substantially parallel to one another and are substantially straight/horizontal between the entry surface 650A and exit surface 654A. The shape of light guide 624A may be referred to as a being straight or flat.

The height H and width W of the exit surface 654A may be approximately equal to corresponding dimensions of the entry surface 650A. Further, the surface area of the entry surface 650A (and entry surfaces 650B-G described below) may be substantially equal to the light emitting portion of the light source 620. The ratio of the surface areas of the entry surface 650A and exit surface 654A affect the degree of divergence of the light leaving the exit surface 650A. The relationship may be defined by the Lagrange invariant, provided in Eq. (2).

$${A}_{entry} \times {Ω}_{entry} = {A}_{exit} \times {Ω}_{exit} \qquad Eq . ( 2 )$$

In Eq. (2), A_{entry} is the surface area of the entry surface 650A, Aₑₓᵢₜ is the surface are of the exit surface 654A, Ω_{entry} is the solid angle of light coupled to the entry surface 650A, and Ωₑₓᵢₜ is the solid angle of the light leaving the exit surface 654A. The solid angle, Ω, is a three-dimensional angle that defines an angular volume. In the context of the exit surface 654A, the exit solid angle Ωₑₓᵢₜ provides an indication of the spread or divergence of the light leaving the exit surface 654A. Rearranging Eq. (2), the exit solid angle Ωₑₓᵢₜ may be written in terms of the remaining terms.

$${Ω}_{exit} = \frac{{A}_{entry}}{{A}_{exit}} \times {Ω}_{entry} \qquad Eq . ( 3 )$$

As indicated by Eq. (3), in examples where the surface area (Aₑₓᵢₜ) of the exit surface 654A is less than the surface area (A_{entry}) of the entry surface 650A, the exit solid angle Ωₑₓᵢₜ is larger than the entry solid angle Ω_{entry}. This arrangement causes the light transmitted from the exit surface 654A to diverge, or to be projected more broadly than light entering the light guide 624A at the entry surface 650A. Thus, the projected light may illuminate a larger portion of the airway. Conversely, in examples where Aₑₓᵢₜ is larger than A_{entry}, light transmitted from the exit surface 654A converges, or is projected more narrowly than light entering the example light guide 624A at the entry surface 650A. In such examples, the projected light may illuminate a smaller portion of the airway.

Returning to example light guide 624A depicted in FIG. 6A, the exit surface 654A is substantially equal to the entry surface 650A (the height H is substantially equal for both surfaces and width W is substantially equal for both surfaces). Light transmitted from the exit surface 654A neither converges nor diverges, relative to the light entering the example light guide 624A at the entry surface 650A.

FIG. 6B depicts an example light guide 624B in which the surface area of the exit surface 654B is larger than the surface area of the entry surface 650B. Top surface 652B and bottom surface 656B are arranged to form a funnel shape, where the top surface 652B and bottom surface 656B diverge or expand away from the center C1 of the entry surface 650B, in the proximal-to-distal direction. This arrangement increases the height H of the exit surface 654B relative to the height H of exit surface 654A. The side surfaces 660B and 662B remain substantially parallel to one another, such that the width W of the exit surface 654B is substantially the same as the width W of exit surface 654A. Light guide 624B may transmit light from the exit surface 654B that is projected more broadly than for example light guide 624A. In addition, the outwardly directed top surface 652B and bottom surface 656B may promote total internal reflection along those surfaces, since a greater portion of the light directed toward the top surface 652B and bottom surface 656B from the light source 620 may be incident at angles larger than the critical angle.

FIG. 6C depicts a further variation of example light guide 624A, in which the exit surface 654C of example light guide 624C is both wider (larger width W) and taller (larger height H) than the exit surface 654A. The width W may be increased by orienting the side walls 660C and 662C to expand or diverge between the entry surface 650C and exit surface 654C, in a proximal-to-distal direction. Example light guide 624C may transmit light from the exit surface 654C that is projected even more broadly than for example light guides 624A and 624B. Similar to example light guide 624B, example light guide 624C is substantially funnel-shaped.

FIG. 6D depicts an example light guide 624D in which the top and bottom surfaces 652D, 656D expand in the proximal-to-distal direction from the center C1 of the entry surface 650D in the proximal region 658D, to form a funnel shape in the proximal region 658D. However, the bottom surface 656D includes a corner or bend in the middle portion 659D such that the bottom surface 656D becomes substantially flat or horizontal between the middle portion 659D and the exit surface 654D. The side surfaces 660D and 662D remain parallel and straight between the entry surface 650D and the exit surface 654D, with the width W of the exit surface 654D remaining substantially equal to the width W of the exit surface 654A. The height H of the exit surface 654D is larger than height H of exit surface 654A, resulting in an exit surface 654D with a larger surface area than for example light guide 624A. Relative to example light guide 624A, example light guide 624D may transmit light from the exit surface 654D that is projected more broadly than for example light guide 624A, but that may not be projected as broadly as example light guide 624C.

FIG. 6E depicts an example light guide 624E that may be similar to, or the same as, light guide 524 depicted in FIG. 5. The surface area of the exit surface 654E may be substantially equal to the surface area of the entry surface 650E, which may have negligible effect on the divergence of light at the exit surface 654E relative to the entry surface 650E. As described above, the center C2 of the exit surface 654E is shifted vertically from center C1 of the entry surface 650E, such that the exit surface 654E is farther from the camera (depicted in FIG. 5). This arrangement may reduce the transmission of escaped light from the light guide 624E to the camera and/or internal scattering that may result in unwanted optical effects in the airway image. As compared to the light guide 624A, the light guide 624E provides a more uniform illumination of the anatomy in the FOV of the camera, which allows for a higher quality image to be captured. Additionally, the offset centers of the exit surface 654E and the entry surface 650E also reduced the amount of glare in the airway image due to reflections of the light, such as from the blade.

FIG. 6F depicts an example light guide 624F that is similar to light guide 624E, but with an exit surface 654F that is smaller than exit surface 654E. The bottom surface 656F of light guide 624F is oriented more steeply towards the center C1 than the bottom surface 656E of light guide 624E. The center C2 of the exit surface 654F is offset from center C1 of the entry surface 650F a substantially similar distance as in light guide 624E. This arrangement reduces the height H of exit surface 654F relative to entry surface 650F, and relative to exit surface 654E of FIG. 6E. Therefore, the light guide 624F may transmit light from the exit surface 654F that is projected more broadly than for light guide 624E.

FIG. 6G depicts a light guide 624G in which the height H of the exit surface 654G is further reduced from the height H of exit surfaces 654E-F, which further reduces the surface area of the exit surface 654G. The light guide 624G may therefore transmit light from the exit surface 654G that is projected even more broadly than for light guides 624E-F. In examples, the smaller exit surface 654G may broaden the projection of light from the exit surface 654G to the extent that the occurrence of glare and/or other unwanted optical effects in the airway image may be increased. Example light guide 624G may be considered substantially cone-shaped.

In some examples, the center C2 of the exit surface 654G is substantially horizontally aligned with center C1 of the entry surface 650G. In other examples, the exit surface 654G may be positioned with center C2 above or below center C1 of the entry surface 650G, in order to achieve a desired effect on illumination of the airway.

In further examples, in addition to varying the surface area of exit surfaces 654A-G, the light guides 624A-G depicted in FIGS. 6A-G may be further modified to affect illumination of the airway. In one example, texturing may be added to the exit surfaces 654A-G to cause diffusion of the light and potentially increase uniformity of the airway illumination. In other examples, the exit surfaces 654A-G and/or entry surfaces 650A-G may be shaped, such as in a concave or convex shape, to further steer light toward the camera FOV and/or away from surfaces of the VL blade. In still other examples, the entry surfaces 650A-G and/or exit surfaces 654A-G may be tilted to effectively direct light (e.g., to improve total internal reflection, to direct light towards the FOV, etc.).

FIGS. 7A-D depict example light guides 724A-D that illustrate a selection of possible shapes and curvatures of surfaces of the light guides 724A-D. FIG. 7A depicts an example light guide 724A with a top surface 752A and bottom surface 756A that follow parabolic curves, and which may be referred to as a parabolic concentrator. The top surface 752A may follow a portion of a first parabolic curve with a first parabolic axis, and the bottom surface 756A may follow a portion of a second parabolic curve with a second parabolic axis, different from the first parabolic axis. The example light guide 724A may promote total internal reflection, since the top surface 752A and bottom surface 756A are oriented in a direction that may increase the incident angle of the light directed through the entry surface 750A from a light source (such as light source 520, depicted in FIG. 5). In some examples, each side surface (e.g., side surfaces 660A and 662A) of the example light guide 724A may also follow parabolic curves or may have other shapes.

As described by Eq. (3), since the surface area of the exit surface 754A is larger than the surface area of the entry surface 750A, light received through the entry surface 750A may be projected more narrowly from the exit surface 754A. The example light guide 724A may be further modified as described, to control other aspects of illumination, such as with a shaped entry surface 750A and/or exit surface 754A, texturing applied on the interior of the exit surface 754A, etc.

FIG. 7B depicts an example light guide 724B, where the top surface 752B and bottom surface 756B are substantially straight and form a funnel shape between the entry surface 750B and exit surface 754B. Side surfaces of the example light guide 724B may also be oriented to form a funnel shape or may conform to another curve or shape. Similar to example light guide 724A, the shape of the example light guide 724B may promote total internal reflection within the light guide 724B. In addition, the larger surface area of the exit surface 754B, relative to the surface area of the entry surface 750B, may narrow the projection of light received through the entry surface 750B and transmitted from the exit surface 754B.

FIG. 7C depicts an example light guide 724C, where, similar to example light guide 724B, the top surface 752C and bottom surface 756C are substantially straight and form a funnel shape between the entry surface 750C and exit surface 754C. However, the length of the bottom surface 756C may be extended to tilt the exit surface 754C at an oblique angle. Due to refraction at the exit surface 754C, the angle of the exit surface 754C directs the light transmitted from the exit surface 754C in the direction of the bottom surface 756C, such as toward the camera and away from the flange of the VL blade. The exit surface 754C may be designed with a different angle of tilt (e.g., a steeper or shallower angle) in order to direct the light transmitted from the exit surface 754C as desired. Further, the relatively larger surface area of the exit surface 754C causes the light transmitted from the exit surface 754C to be projected more narrowly than the light received through the entry surface 750C.

FIG. 7D depicts an example light guide 724D designed with surfaces that are unique from one another. The example light guide 724D may be referred to, in some examples, as a free-form optical element. The top surface 752D and bottom surface 756D may have dissimilar or substantially dissimilar shapes, may not be symmetric with one another, and may or may not follow commonly used or conventional curves (such as a straight line, a parabolic curve, etc.). In some examples, the shapes of the top surface 752D and/or bottom surface 756D may be based on a combination of two or more curves joined together to form the shape of the top surface 752D and/or bottom surface 756D. For instance, a first portion of the top surface 725D may follow a parabolic curve and may be joined to a second portion that may follow an exponential curve. In other examples, the top surface 752D and bottom surface 756D may be designed with fully custom curves or shapes, rather than a single or combination of conventional curves. In examples, the top surface 752D and bottom surface 756D may be shaped to achieve a desired level of total internal reflection or other optical metric, to fit around an external element or conform to a desired shape within the arm (such as example arm 532), and/or for another purpose.

In addition, the exit surface 754D may be based on commonly used or conventional curves, a combination of curves, or may be a custom shape or curve. Similar to the top surface 752D and bottom surface 756D, the side surfaces may or may not be symmetrical with one another and/or may be based on a single curve, combination of curve, or may be a custom curve.

As described above, the example light guides 724A-D may be further modified to affect illumination of the airway. For example, the example light guides 724A-D may include texturing on the interior of the exit surfaces 754A-D to cause diffusion of the light transmitted through the exit surfaces 754A-D. Additionally, the shape of entry surfaces 750A-D and/or exit surfaces 754A-D may be convex or concave, or other modifications may be made to the surfaces of the example light guides 724A-D to affect the illumination of the airway.

Other optical elements may be included on the exterior of the light guides 724A-D to further modify the light transmitted from the exit surfaces 754A-D. For example, portions of the light guides 724A-D (and light guides 624A-G, 524, etc.) may be designed to direct light through an external lens that further shapes or directs the light onto the camera FOV for brighter and/or more uniform illumination. In one example, a lens may be associated with the window of the blade channel (such as lenses 114 and 214). Accordingly, while the unibody plug remains the same, the optical path may change when different blades are attached to the laryngoscope. In other examples, an external lens or other optical elements may be associated with other portions of the blade (such as blade 106), arm (such as arms 132, 232, 432), and/or with other portions or structures associated with the light guides 724A-D.

Those skilled in the art will recognize that the methods and systems of the present disclosure may be implemented in many manners and as such are not to be limited by the foregoing aspects and examples. In other words, functional elements being performed by a single or multiple components. In this regard, any number of the features of the different aspects described herein may be combined into single or multiple aspects, and alternate aspects having fewer than or more than all of the features herein described are possible. Functionality may also be, in whole or in part, distributed among multiple components, in manners now known or to become known.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A video laryngoscope comprising:
a display;
a handle;
an arm extending distally from the handle;
a camera positioned at a distal end of the arm;
a light source positioned at the distal end of the arm; and
a unibody plug positioned at the distal end of the arm, the unibody plug comprising:
a light guide formed of a solid transparent material having an entry surface at a proximal end of the light guide and an exit surface at a distal end of the light guide, wherein the entry surface is adjacent the light source; and
a frame that defines an air gap around at least 70% of a length of the light guide.

2. The video laryngoscope of claim 1, wherein the frame further defines a cavity for receiving the camera.

3. The video laryngoscope of claim 1, wherein the frame and the light guide are formed from the same transparent material.

4. The video laryngoscope of claim 1, wherein a surface area of the entry surface is less than a surface area of the exit surface.

5. The video laryngoscope of claim 1, wherein a center of the exit surface is positioned anteriorly from a center of the entry surface.

6. The video laryngoscope of claim 1, wherein at least one of the entry surface or the exit surface is textured to cause diffusion of light from the light source.

7. The video laryngoscope of claim 1, wherein at least one of the entry surface or the exit surface is formed as a concave or convex lens.

8. A unibody plug for guiding light emitting from a video laryngoscope, the unibody plug comprising:
a light guide, formed of a solid transparent material, having an entry surface at a proximal end of the light guide and an exit surface at a distal end of the light guide, wherein the entry surface is adjacent a light source of the video laryngoscope; and
a frame, formed of the solid transparent material, that defines an air gap around at least 70% of a length of the light guide.

9. The unibody plug of claim 8, wherein the air gap is around at least 90% of the length of the light guide.

10. The unibody plug of claim 8, wherein the frame further defines a cavity for receiving a camera of the video laryngoscope.

11. The unibody plug of claim 8, wherein a center of the exit surface is positioned anteriorly from a center of the entry surface.

12. The unibody plug of claim 11, wherein a surface area of the entry surface is substantially the same as a surface area of the exit surface.

13. The unibody plug of claim 11, wherein a width and height of the entry surface is substantially the same as a width and height of the exit surface.

14. The unibody plug of claim 8, wherein the air gap is filled with a material that has a refractive index different than the transparent material of the light guide.

15. A video laryngoscope comprising:
a display;
a handle;
an arm extending distally from the handle;
a camera positioned at a distal end of the arm;
a light source positioned at the distal end of the arm; and
a unibody plug positioned at the distal end of the arm, the unibody plug comprising:
a light guide formed of a solid transparent material, the light guide comprising:
an entry surface at a proximal end of the light guide, the entry surface having a first center and a first surface area;
an exit surface at the distal end of the light guide, the exit surface having:
a second center positioned anteriorly from the first center of the entry surface; and
a second surface area that is substantially the same as the first surface area of the entry surface; and
a frame formed of the solid transparent material, the frame defining:
an air gap around at least 90% of a length of the light guide; and
a cavity that receives the camera.
